(19) United States
(12) Patent Application Publication
(10) Pub. No.: US 2012/0164506 A1
(43) Pub. Date: Jun. 28, 2012

(54) Ester Based Heat Transfer Fluid Useful as a Coolant for Electric Vehicles

(75) Inventors: Sandra G. Claeys, Lovendegem (BE); Serge S. Lievens, Merelbeke (BE); Paul Van De Ven, Dilbeek (BE); Zhen Zhou, Emeryville, CA (US); Stephen J. Miller, San Francisco, CA (US); Ryan J. Schexnaydre, Richmond, CA (US); Saleh Elomari, Fairfield, CA (US)

(73) Assignee: Chevron U.S.A. Inc., San Ramon, CA (US)

(21) Appl. No.: 12/978,019

(22) Filed: Dec. 23, 2010

(51) Int. Cl. H01M 10/50; C09K 5/04; C07C 69/34

(52) U.S. Cl. 429/120; 560/190; 252/67

(57) ABSTRACT

Provided is a heat transfer fluid formulation comprising at least one diester or triester species having ester links on adjacent carbons. The formulation exhibits an excellent balance of dielectric and heat transfer properties, and is useful as a coolant for electric vehicles.

### BACKGROUND

1. Technical Field

Provided is an ester based heat transfer fluid. More specifically, the heat transfer fluid is comprised of an ester species having ester links on adjacent carbons, and is useful as a coolant for electric vehicles.

2. Description of the Related Art

An increased interest is observed towards electric vehicle technology. This interest is driven by more severe emission regulations, the challenge to reduce the dependency of oil and the need to improve energy efficiency of transportation. Globally, the trend is towards more efficient vehicles that have good fuel economy and less emissions. Particular emphasis is on reducing CO₂ emissions. Accordingly, the focus is on electric vehicles. Some governments have introduced incentives for producing and purchasing electric vehicles.

Despite the attractive benefits electric vehicles can provide, the introduction into the market and their production has until now been very limited because of certain technical barriers and associated costs that need to be resolved. One of these challenges is the optimization of the thermal management of the electric drive systems. The optimum operating temperature range of system components like the battery pack differs significantly from that of the electromotor and power electronics and they have an important impact on the performance and the life of these critical parts. Breakdown of these parts would not only result in an increase of the vehicles maintenance cost and loss of efficiency, but in worse cases no longer guarantee safe operation of the vehicle.

An optimized thermal management system requires efficient cooling and heating methods which are able to keep the temperature constant within the optimum temperature range of the electric drive components. In applications today, air or liquids are most often used as a heat transfer medium. As the liquid, water/glycol mixtures, refrigerants and oils are known and described in the literature. Whereas air has the advantages of lower cost, less maintenance and lower weight compared with liquid cooling, the latter has better heat transfer properties. Within the group of liquids, a difference in heat transfer properties is observed. Water/glycol (aqueous based) mixtures have much higher heat transfer properties as compared with other non aqueous based liquids such as oils (e.g. silicone oils), chlorofluorocarbons and other organic liquids (e.g. alkyl benzenes). Water/glycol mixtures are therefore often used as indirect contact liquids transferring the heat by running through tubes and plates which are in contact with the electronic parts. Direct contact with water/glycol mixtures is avoided because of its high electrical conductivity resulting in electricity leakages and power losses to the heat transfer fluid.

For those applications where water/glycol coolants have been evaluated, the electrical conductivities are kept low by the use of ion exchange resins or other ion exchange methods which greatly reduce the presence of ions. Other methods which have been used to keep electrical conductivities low in water/glycol mixtures are the selection of certain corrosion inhibitors such as non ionic compounds and/or additives that increase the oxidative stability of the glycol in the base fluid or the use of certain types of glycol base fluids with higher oxidative stability. The non aqueous based liquids have dielectric (electric insulating) properties characterized by very low electrical conductivities. The less effective heat transfer properties of these fluids are in more recent developments improved by dispersion of phase change materials or highly heat conductive materials, or combination with a base fluid with better heat transfer properties. In order to be suitable for cold climates and seasons, both aqueous and non aqueous based heat transfer fluids have antifreeze requirements.

The industry is constantly searching for a coolant that can meet the dielectric properties, thermal conductivity and specific heat requirements for an electric vehicle. Such a coolant, which is also environmentally friendly and can offer good cold weather operation, would be of great benefit to the electric vehicle industry.

### SUMMARY

The subject of the resent invention is a non aqueous base fluid with dielectric properties which can be used as a heat transfer fluid for applications with low electric conductive requirements, such as for electric drive systems. The heat transfer fluid is ester based, and is specifically comprised of a diester or triester having ester links on adjacent carbons. The heat transfer properties of the ester based fluids are suitable for use as a heat transfer fluid. Additional advantages offered compared with other non aqueous dielectric heat transfer fluids are low environmental impact, low flammability and cost efficiency.

In another embodiment, an electric vehicle is provided in which the coolant used therein is comprised of the present diester or triester having ester links on adjacent carbons. The coolant can be used in the battery, the electric motor cooling loop, or as the coolant for the fuel cell, or in any combination or in all of the foregoing.

In another embodiment, a process for operation of an electric vehicle is provided wherein the coolant used in the vehicle comprises a diester or triester having ester links on adjacent carbons.

Among other factors, the present diester and triester based heat transfer fluid provides a heat transfer fluid which is biodegradable and environmentally friendly, has low flammability and is therefore safe. Yet, the present ester based fluid has the dielectric, thermal conductivity and specific heat properties necessary to allow the use as a coolant, and more particularly is well-suited for use in an electric vehicle.

### DETAILED DESCRIPTION OF THE EMBODIMENTS

As used herein, the following terms have the following meanings unless expressly stated to the contrary. The test methods noted below are those generally used, but any other test method which gives equivalent results can be used.

“Pour point,” as defined herein, represents the lowest temperature at which a fluid will pour or flow. See, e.g., ASTM International Standard Test Methods D 5950-02 (Reapproved 2007), Standard Test Method for Pour Point of Petroleum Products (Automatic Tilt Method).

“Cloud point,” as defined herein, represents the temperature at which a fluid begins to phase separate due to crystal formation. The test method for determining cloud point is ASTM-D5773-10, Standard Test Method for Cloud Point of Petroleum Products (Constant Cooling Rate Method).

Kinematic Viscosity: ASTM D445-10, Standard Test Method for Kinematic Viscosity of Transparent and Opaque Liquids (and Calculation of Dynamic Viscosity)

With respect to describing molecules and/or molecular fragments herein, “Rn,” where “n” is an index, refers to a hydrocarbon group, wherein the molecules and/or molecular fragments can be linear and/or branched.

As defined herein, “Cn,” where “n” is an integer, describes a hydrocarbon molecule or fragment (e.g., an alkyl group) wherein “n” denotes the number of carbon atoms in the fragment or molecule.

The prefix “bio,” as used herein, refers to an association with a renewable resource of biological origin, such as resource generally being exclusive of fossil fuels. The term “internal olefin,” as used herein, refers to an olefin (i.e., an alkene) having a non-terminal carbon-carbon double bond (C—C). This is in contrast to “α-olefins” which do bear a terminal carbon-carbon double bond.

The term “comprising” means including the elements or steps that are identified following that term, but any such elements or steps are not exhaustive, and an embodiment can include other elements or steps.

One embodiment is directed to a heat transfer fluid composition comprising (a) a diester or triester-based heat transfer fluid derived from a biomass precursor and/or low value Fischer-Tropsch (FT) olefins and/or alcohols. In some embodiments, such diester or triester-based heat transfer fluids are derived from FT olefins and fatty (carboxylic) acids. In these or other embodiments, the fatty acids can be from a bio-based source (i.e., biomass, renewable source) or can be derived from FT alcohols via oxidation.

### Diester Heat Transfer Fluid Compositions

In some embodiments, the present invention is generally directed to diester-based heat transfer fluid compositions comprising a quantity of diester species having the following chemical structure:

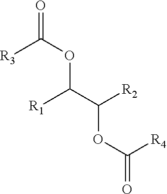

where R₁, R₂, R₃, and R₄ are the same or independently selected from a C₂ to C₁₇ carbon fragment, i.e., a hydrocarbon group having from 2 to 17 carbon atoms.

Regarding the above-mentioned diester species, selection of R₁, R₂, R₃, and R₄ can follow any or all of several criteria. For example, in some embodiments, R₁, R₂, R₃, and R₄ are selected such that the kinematic viscosity of the composition at a temperature of 100° C. is typically 3 mm²/sec or greater. In some or other embodiments, R₁, R₂, R₃, and R₄ are selected such that the pour point of the resulting heat transfer fluid is −10° C. or lower, −25° C. or lower; or even −40° C. or lower. In some embodiments, R₁ and R₂ are selected to have a combined carbon number (i.e., total number of carbon atoms) of from 6 to 14. In these or other embodiments, R₃ and R₄ are selected to have a combined carbon number of from 10 to 34. Depending on the embodiment, such resulting diester species can have a molecular mass between 340 atomic mass units (a.m.u.) and 780 a.m.u.

In some embodiments, such above-described compositions are substantially homogeneous in terms of their diester component. In some or other embodiments, the diester component of such compositions comprises a variety (i.e., a mixture) of diester species.

In some embodiments, the diester-based heat transfer fluid composition comprises at least one diester species derived from a C₈ to C₁₆ olefin and a C₂ to C₁₈ carboxylic acid. Typically, the diester species are made by reacting each -OH group (on the intermediate) with a different acid, but such diester species can also be made by reacting each —OH group with the same acid.

In some of the above-described embodiments, the diester-based heat transfer fluid composition comprises a diester species selected from the group consisting of decanoic acid 2-decanoyloxy-1-hexyl-octyl ester and its isomers, tetradecanoic acid-1-hexyl-2-tetradecanoyloxy-octyl esters and its isomers, dodecanoic acid 2-dodecanoyloxy-1-hexyl-octyl ester and its isomers, hexanoic acid 2-hexanoyloxy-1-hexy-octyl ester and its isomers, octanoic acid 2-octanoyloxy-1-hexyl-octyl ester and its isomers, hexanoic acid 2-hexanoyloxy-1-pentyl-heptyl ester and isomers, octanoic acid 2-octanoyloxy-1-pentyl-heptyl ester and isomers, decanoic acid 2-decanoyloxy-1-pentyl-heptyl ester and isomers, decanoic acid-2-cecanoyloxy-1-pentyl-heptyl ester and its isomers, dodecanoic acid-2-dodecanoyloxy-1-pentyl-heptyl ester and isomers, tetradecanoic acid 1-pentyl-2-tetradecanoyloxy-heptyl ester and isomers, tetradecanoic acid 1-butyl-2-tetradecanoyloxy-hexy ester and isomers, dodecanoic acid-1-butyl-2-dodecanoyloxy-hexyl ester and isomers, decanoic acid 1-butyl-2-decanoyloxy-hexyl ester and isomers, octanoic acid 1-butyl-2-octanoyloxy-hexyl ester and isomers, hexanoic acid 1-butyl-2-hexanoyloxy-hexyl ester and isomers, tetradecanoic acid 1-propyl-2-tetradecanoyloxy-pentyl ester and isomers, dodecanoic acid 2-dodecanoyloxy-1-propyl-pentyl ester and isomers, decanoic acid 2-decanoyloxy-1-propyl-pentyl ester and isomers, octanoic acid 1-2-octanoyloxy-1-propyl-pentyl ester and isomers, hexanoic acid 2-hexanoyloxy-1-propyl-pentyl ester and isomers, and mixtures thereof.

The above-described esters can also be used as blending stocks. As such, esters with higher pour points can also be used as blending stocks with other heat transfer fluids, such as other coolant oils, since they are very soluble in hydrocarbons and hydrocarbon-based oils.

### Methods of Making Diester Heat Transfer Fluids

As mentioned above, the present invention is additionally directed to methods of making the above-described heat transfer fluid compositions. The methods employed in the making of the diesters are further described in U.S. Patent Application Publications 2009/0159837 and 2009/0198075, which publications are incorporated by reference herein in their entirety.

In some embodiments, processes for making the above-mentioned diester species, typically having the desired dielectric and thermal conductivity properties, comprise the following steps: epoxidizing an olefin (or quantity of olefins) having a carbon number of from 8 to 16 to form an epoxide comprising an epoxide ring; opening the epoxide ring to form a diol; and esterifying (i.e., subjecting to esterification) the diol with an esterifying species to form a diester species, wherein such esterifying species are selected from the group consisting of carboxylic acids, acyl acids, acyl halides, acyl anhydrides, and combinations thereof; wherein such esterifying species have a carbon number from 2 to 18; and wherein the diester species have a viscosity of 3 mm²/sec or more at a temperature of 100° C.

Furthermore, the diester species can be prepared by epoxidizing an olefin having from about 8 to about 16 carbon atoms to form an epoxide comprising an epoxide ring. The epoxidized olefin is reacted directly with an esterifying species to form a diester species, wherein the esterifying species is selected from the group consisting of carboxylic acids, acyl halides, acyl anhydrides, and combinations thereof, wherein the esterifying species has a carbon number of from 2 to 18, and wherein the diester species has a viscosity and a pour point suitable for use as an heat transfer fluid.

In some embodiments, where a quantity of such diester species is formed, the quantity of diester species can be substantially homogeneous, or it can be a mixture of two or more different such diester species.

In some such above-described method embodiments, the olefin used is a reaction product of a Fischer-Tropsch process. In these or other embodiments, the carboxylic acid can be derived from alcohols generated by a Fischer-Tropsch process and/or it can be a bio-derived fatty acid.

In some embodiments, the olefin is an a-olefin (i.e., an olefin having a double bond at a chain terminus). In such embodiments, it is usually necessary to isomerize the olefin so as to internalize the double bond. Such isomerization is typically carried out catalytically using a catalyst such as, but not limited to, crystalline aluminosilicate and like materials and aluminophosphates. See, e.g., U.S. Pat. Nos. 2,537,283; 3,211,801; 3,270,085; 3,327,014; 3,304,343; 3,448,164; 4,593,146; 3,723,564 and 6,281,404; the last of which claims a crystalline aluminophosphate-based catalyst with 1-dimensional pores of size between 3.8 Å and 5 Å.

As an example of such above-described isomerizing, Fischer-Tropsch alpha olefins (α-olefins) can be isomerized to the corresponding internal olefins followed by epoxidation. The epoxides can then be transformed to the corresponding diols via epoxide ring opening followed by di-acylation (i.e., di-esterification) with the appropriate carboxylic acids or their acylating derivatives. It is typically necessary to convert alpha olefins to internal olefins because diesters of alpha olefins, especially short chain alpha olefins, tend to be solids or waxes. “Internalizing” alpha olefins followed by transformation to the diester functionalities introduces branching along the chain which reduces the pour point of the intended products. The ester groups with their polar character would further enhance the viscosity of the final product. Adding ester branches will increase the carbon number and hence viscosity. It can also decrease the associated pour and cloud points. It is typically preferable to have a few longer branches than many short branches, since increased branching tends to lower the viscosity index (VI).

Regarding the step of epoxidizing (i.e., the epoxidation step), in some embodiments, the above-described olefin (in one embodiment an internal olefin) can be reacted with a peroxide (e.g., H₂O₂) or a peroxy acid (e.g., peroxyacetic acid) to generate an epoxide. See, e.g., D. Swern, in Organic Peroxides Vol. II, Wiley-Interscience, New York, 1971, pp. 355-533; and B. Plesnicar, in Oxidation in Organic Chemistry, Part C, W. Trahanovsky (ed.), Academic Press, New York 1978, pp. 221-253. Olefins can be efficiently transformed to the corresponding diols by highly selective reagent such as osmium tetra-oxide (M. Schroder, Chem. Rev. vol. 80, p. 187, 1980) and potassium permanganate (Sheldon and Kochi, in Metal-Catalyzed Oxidation of Organic Compounds, pp. 162-171 and 294-296, Academic Press, New York, 1981).

Regarding the step of epoxide ring opening to the corresponding diol, this step can be acid-catalyzed or based-catalyzed hydrolysis. Exemplary acid catalysts include, but are not limited to, mineral-based Brönsted acids (e.g., HCl, H₂SO₄, H₃PO₄, perhalogenates, etc.), Lewis acids (e.g., TiCl₄ and AlCl₃) solid acids such as acidic aluminas and silicas or their mixtures, and the like. See, e.g., Chem. Rev. vol. 59, p. 737, 1959; and Angew. Chem. Int. Ed., vol. 31, p. 1179, 1992. Based-catalyzed hydrolysis typically involves the use of bases such as aqueous solutions of sodium or potassium hydroxide.

Regarding the step of esterifying (esterification), an acid is typically used to catalyze the reaction between the -OH groups of the diol and the carboxylic acid(s). Suitable acids include, but are not limited to, sulfuric acid (Munch-Peterson, Org. Synth., V, p. 762, 1973), sulfonic acid (Allen and Sprangler, Org. Synth., III, p. 203, 1955), hydrochloric acid (Eliel et al., Org. Synth., IV, p. 169, 1963), and phosphoric acid (among others). In some embodiments, the carboxylic acid used in this step is first converted to an acyl chloride (via, e.g., thionyl chloride or PCl₃). Alternatively, an acyl chloride could be employed directly. Wherein an acyl chloride is used, an acid catalyst is not needed and a base such as pyridine, 4-dimethylaminopyridine (DMAP) or triethylamine (TEA) is typically added to react with an HCl produced. When pyridine or DMAP is used, it is believed that these amines also act as a catalyst by forming a more reactive acylating intermediate. See, e.g., Fersh et al., J. Am. Chem. Soc., vol. 92, pp. 5432-5442, 1970; and Hofle et al., Angew. Chem. Int. Ed. Engl., vol. 17, p. 569, 1978.

Regardless of the source of the olefin, in some embodiments, the carboxylic acid used in the above-described method is derived from biomass. In some such embodiments, this involves the extraction of some oil (e.g., triglyceride) component from the biomass and hydrolysis of the triglycerides of which the oil component is comprised so as to form free carboxylic acids.

### Triester Heat Transfer Fluid Compositions

In some embodiments, the present, invention is generally directed to triester-based heat transfer fluid compositions comprising a quantity of triester species having the following chemical structure:

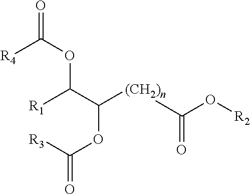

wherein R₁, R₂, R₃, and R₄ are the same or independently selected from C₂ to C₂₀ hydrocarbon groups (groups with a carbon number from 2 to 20), and wherein “n” is an integer from 2 to 20.

Regarding the above-mentioned triester species, selection of R₁, R₂, R₃, and R₄, and n can follow any or all of several criteria. For example, in some embodiments, R₁, R₂, R₃, and R₄ and n are selected such that the kinematic viscosity of the composition at a temperature of 100° C. is typically 3 mm²/sec or greater. In some or other embodiments, R₁, R₂, R₃, and R₄ and n are selected such that the pour point of the resulting heat transfer fluid is −10° C. or lower, e.g., −25° C. or even −40° C. or lower. In some embodiments, R₁ is selected to have a total carbon number of from 6 to 12. In these or other embodiments, R₂ is selected to have a carbon number of from 1 to 20. In these or other embodiments, R₃ and R₄ are selected to have a combined carbon number of from 4 to 36. In these or other embodiments, n is selected to be an integer from 5 to 10. Depending on the embodiment, such resulting triester species can typically have a molecular mass between 400 atomic mass units (a.m.u.) and 1100 a.m.u, and more typically between 450 a.m.u. and 1000 a.m.u.

In some embodiments, such above-described compositions are substantially homogeneous in terms of their triester component. In some or other embodiments, the triester component of such compositions comprises a variety (i.e., a mixture) of such triester species. In these or other embodiments, such above-described heat transfer fluid compositions further comprise one or more triester species.

In some of the above-described embodiments, the triester-based heat transfer fluid composition comprises one or more triester species of the type 9,10-bis-alkanoyloxy-oetadecanoic acid alkyl ester and isomers and mixtures thereof, where the alkyl is selected from the group consisting of methyl, ethyl, propyl, butyl, pentyl, hexyl, heptyl, octyl, nonyl, decyl, undecyl, dodecyl, tridecyl, tetradecyl, pentadecyl, hexadecyl, and octadecyl; and where the alkanoyloxy is selected from the group consisting of ethanoyloxy, propanoyoxy, butanoyloxy, pentanoyloxy, hexanoyloxy, heptanoyloxy, octanoyloxy, nonaoyloxy, decanoyloxy, undacanoyloxy, dodecanoyloxy, tridecanoyloxy, tetradecanoyloxy, pentaclecanoyloxy, hexadeconoyloxy, and octadecanoyloxy, 9,10-bis-hexanoyloxy-octadecanoic acid hexyl ester and 9,10-bis-decanoyloxy-octadecanoic acid decyl ester are exemplary such triesters.

It is worth noting that the above-described triesters and their compositions can be used as heat transfer fluids by themselves, but can also be used as blending stocks. As such, esters with higher pour points can also be used as blending stocks with other heat transfer fluids since they are very soluble in hydrocarbons and hydrocarbon-based oils.

### Methods of Making Triester Heat Transfer Fluids

As mentioned above, the present invention is additionally directed to methods of making the above-described heat transfer fluid compositions and/or the triester compositions contained therein. Such a method is described in U.S. Pat. No. 7,544,645, which is incorporated herein by reference in its entirety.

In some embodiments, processes for making the above-mentioned triester-based compositions, typically having the desired dielectric and thermal conductivity properties, comprise the following steps: esterifying (i.e., subjecting to esterification) a mono-unsaturated fatty acid (or quantity of mono-unsaturated fatty acids) having a carbon number of from 16 to 22 with an alcohol to form an unsaturated ester (or a quantity thereof); epoxidizing the unsaturated ester to form an epoxy-ester species comprising an epoxide ring; opening the epoxide ring of the epoxy-ester species to form a dihydroxy-ester: and esterifying the dihydroxy-ester with an esterifying species to form a triester species, wherein such esterifying species are selected from the group consisting of carboxylic acids, acyl halides, acyl anhydrides, and combinations thereof; and wherein such esterifying species have a carbon number of from 2 to 19. Generally, heat transfer fluid compositions made by such methods and comprising such triester species have a viscosity of 3 mm²/sec or more at a temperature of 100° C. and they typically have a pour point of less than −20° C., and selection of reagents and/or mixture components is typically made with this objective.

In another embodiment, the method can comprise reducing a monosaturated fatty acid to the corresponding unsaturated alcohol. The unsaturated alcohol is then epoxidized to an epoxy fatty alcohol. The ring of the epoxy fatty alcohol is opened to make the corresponding triol; and then the triol is esterified with an esterifying species to form a triester species, wherein the esterifying species is selected from the group consisting of carboxylic acids, acyl halides, acyl anhydrides, and combinations thereof, and wherein the esterifying species has a carbon number of from 2 to 19. With the foregoing method, the triester would generally have the following structure:

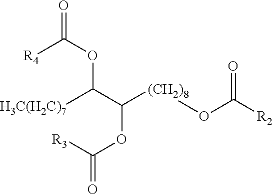

wherein R₂, R₃ and R₄ are typically the same or independently selected from C₂ to C₂₀ hydrocarbon groups, and are typically selected from C₄ to C₁₂ hydrocarbon groups.

In another embodiment, the method can comprise reducing a monosaturated fatty acid to the corresponding unsaturated alcohol; epoxidizing the unsaturated alcohol to an epoxy fatty alcohol; and esterifying the fatty alcohol epoxide with an esterifying species to form a triester species, wherein the esterifying species is selected from the group consisting of carboxylic acids, acyl halides, acyl anhydrides, and combinations thereof, and wherein the esterifying species has a carbon number of from 2 to 19.

In some embodiments, where a quantity of such triester species is formed, the quantity of triester species can be substantially homogeneous, or it can be a mixture of two or more different such triester species. In any such embodiments, such triester compositions can be further mixed with one or more base oils of the type Group I-III. Additionally or alternatively, in some embodiments, such methods further comprise a step of blending the triester composition(s) with one or more diester species.

In some embodiments, such methods produce compositions comprising at least one triester species of the type 9,10-bis-alkanoyloxy-octadecanoic acid alkyl ester and isomers and mixtures thereof where the alkyl is selected from the group consisting of methyl, ethyl, propyl, butyl, pentyl, hexyl, heptyl, octyl, nonyl, decyl, undecyl, dodecyl, tridecyl, tetradecyl, pentadecyl, hexadecyl, and octadecyl; and where the alkanoyloxy is selected from the group consisting of ethanoyloxy, propanoyoxy, butanoyloxy, pentanoyloxy, hexanoyloxy, heptanoyloxy, octanoyloxy, nonaoyloxy, decanoyloxy, undacanoyloxy, dodecanoyloxy, tridecanoyloxy, tetradecanoyloxy, pentadecanoyloxy, hexadeconoyloxy, and octadecanoyloxy. Exemplary such triesters include, but not limited to, 9,10-bis-hexanoyloxy-octadecanoic acid hexyl ester; 9,10-bis-octanoyloxy-octadecanoic acid hexyl ester; 9,10-bis-decanoyloxy-octadecanoic acid hexyl ester; 9,10-bis-dodecanoyoxy-octadecanoic acid hexyl ester; 9,10-bis-hexanoyloxy-octadecanoic acid decyl ester; 9,10-bis-decanoyloxy-octadecanoic acid decyl ester; 9,10-bis-octanoyloxy-octadecanoic acid decyl ester; 9,10-bis-dodecanoyloxy-octadecanoic acid decyl ester; 9,10-bis-hexanoyloxy-octadecanoic acid octyl ester; 9,10-bis-octanoyloxy-octadecanoic acid octyl ester: 9,10-bis-decanoyloxy-octadecanoic acid octyl ester; 9,10-bis-dodecanoyloxy-octadecanoic acid octyl ester; 9,10-bis-hexanoyloxy-octadecanoic acid dodecyl ester; 9,10-bis-octanoyloxy-octadecanoic acid dodecyl ester; 9,10-bis-decanoyloxy-octadecanoic acid dodecyl ester; 9,10-bis-doclecanoyloxy-octadecanoic acid dodecyl ester; and mixtures thereof.

In some such above-described method embodiments, the mono-unsaturated fatty acid can be a bio-derived fatty acid. In some or other such above-described method embodiments, the alcohol(s) can be FT-produced alcohols.

In some such above-described method embodiments, the step of esterifying (i.e., esterification) the mono-unsaturated fatty acid can proceed via an acid-catalyzed reaction with an alcohol using, e.g., H₂SO₄ as a catalyst. In some or other embodiments, the esterifying can proceed through a conversion of the fatty acid(s) to an acyl halide (chloride, bromide, or iodide) or acyl anhydride, followed by reaction with an alcohol.

Regarding the step of epoxidizing (i.e., the epoxidation step), in some embodiments, the above-described mono-unsaturated ester can be reacted with a peroxide (e.g., H₂O₂) or a peroxy acid (e.g., peroxyacetic acid) to generate an epoxy-ester species. See, e.g., D. Swern, in Organic Peroxides Vol. II, Wiley-Interscience, New York, 1971, pp. 355-533; and B. Plesnicar, in Oxidation in Organic Chemistry, Part C, W. Trahanovsky (ed.), Academic Press, New York 1978, pp. 221-253. Additionally or alternatively, the olefinic portion of the mono-unsaturated ester can be efficiently transformed to the corresponding dihydroxy ester by highly selective reagents such as osmium tetra-oxide (M. Schroder, Chem. Rev. vol. 80, p. 187, 1980) and potassium permanganate (Sheldon and Kochi, in Metal-Catalyzed Oxidation of Organic Compounds, pp. 162-171 and 294-296, Academic Press, New York, 1981).

Regarding the step of epoxide ring opening to the corresponding dihydroxy-ester, this step is usually an acid-catalyzed hydrolysis. Exemplary acid catalysts include, but are not limited to, mineral-based Brönsted acids (e.g., HCl, H₂SO₄, H₃PO₄, perhalogenates, etc.), Lewis acids (e.g., TiCl₄ and AlCl₃), solid acids such as acidic aluminas and silicas or their mixtures, and the like. See, e.g., Chem. Rev. vol. 59, p. 737, 1959; and Angew. Chem. Int. Ed., vol. 31, p. 1179, 1992. The epoxide ring opening to the diol can also be accomplished by base-catalyzed hydrolysis using aqueous solutions of KOH or NaOH.

Regarding the step of esterifying the dihydroxy-ester to form a triester, an acid is typically used to catalyze the reaction between the -OH groups of the diol and the carboxylic acid(s). Suitable acids include, but are not limited to, sulfuric acid (Munch-Peterson, Org. Synth., V, p. 762, 1973), sulfonic acid (Allen and Sprangler, Org Synth., III, p. 203, 1955), hydrochloric acid (Eliel et al., Org Synth., IV, p. 169, 1963), and phosphoric acid (among others). In some embodiments, the carboxylic acid used in this step is first converted to an acyl chloride (or another acyl halide) via, e.g., thionyl chloride or PC13. Alternatively, an acyl chloride (or other acyl halide) could be employed directly. Where an acyl chloride is used, an acid catalyst is not needed and a base such as pyridine, 4-dimethylaminopyridine (DMAP) or triethylamine (TEA) is typically added to react with an HCl produced. When pyridine or DMAP is used, it is believed that these amines also act as a catalyst by forming a more reactive acylating intermediate. See, e.g., Fersh et al., J. Am. Chem. Soc., vol. 92, pp. 5432-5442, 1970; and Hofle et al., Angew. Chem. Int. Ed. Engl., vol. 17, p. 569, 1978. Additionally or alternatively, the carboxylic acid could be converted into an acyl anhydride and/or such species could be employed directly.

Regardless of the source of the mono-unsaturated fatty acid, in some embodiments, the carboxylic acids (or their acyl derivatives) used in the above-described methods are derived from biomass. In some such embodiments, this involves the extraction of some oil (e.g., triglyceride) component from the biomass and hydrolysis of the triglycerides of which the oil component is comprised so as to form free carboxylic acids.

In some particular embodiments, wherein the above-described method uses oleic acid for the mono-unsaturated fatty acid, the resulting triester is of the type:

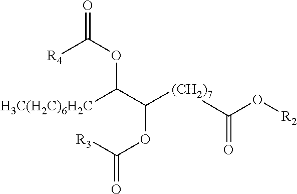

wherein R₂, R₃ and R₄ are typically the same or independently selected from C₂ to C₂₀ hydrocarbon groups, and are more typically selected from C₄ to C₁₂ hydrocarbon groups.

Using a synthetic strategy in accordance with that outlined above, oleic acid can be converted to triester derivatives (9,10-bis-hexanoyloxy-octadecanoic acid hexyl ester) and (9,10-bis-decanoyloxy-octadecanoic acid decyl ester). Oleic acid is first esterified to yield a mono-unsaturated ester. The mono-unsaturated ester is subjected to an epoxidation agent to give an epoxy-ester species, which undergoes ring-opening to yield a dihydroxy ester, which can then be reacted with an acyl chloride to yield a triester product.

The strategy of the above-described synthesis utilizes the double bond functionality in oleic acid by converting it to the diol via double bond epoxidation followed by epoxide ring opening. Accordingly, the synthesis begins by converting oleic acid to the appropriate alkyl oleate followed by epoxidation and epoxide ring opening to the corresponding diol derivative (dihydroxy ester).

Variations (i.e., alternate embodiments) on the above-described heat transfer fluid compositions include, but are not limited to, utilizing mixtures of isomeric olefins and or mixtures of olefins having a different number of carbons. This leads to diester mixtures and triester mixtures in the product compositions.

Variations on the above-described processes include, but are not limited to, using carboxylic acids derived from FT alcohols by oxidation.

Conventional additives can be added to the ester based coolant formulation. Such additives can include ion exchange resins, corrosion inhibitors, oxidative stability additives and phase change materials. Such additives, when used are generally non-ionic in nature, as the additional presence of ionic material would raise the electrical conductivity.

The present heat transfer fluids provide many advantages and have the physical properties to be used as coolants. The present ester based heat transfer fluids are particularly well suited for use as a coolant in an electric vehicle. The coolant can be used in the battery, the electric motor cooling loop, which includes the motor and the power electronics (e.g., inverters and converters), and the fuel cell. The ester based coolant can be used in one of the foregoing components, or in any combination. The coolant can also be used in all three at the same time. The coolant can also be used as an indirect coolant in any fuel cell used in an electric vehicle. The electric vehicle can be a total electric vehicle or a hybrid.

The present fluid coolant comprised of a diester or triester species exhibits an electrical volume resistivity at 25° C. of at least 10¹⁰ ohm-cm, and generally at least 10¹² ohm-cm. The specific heat of the present coolant as exhibited at 20° C. is generally at least 2.00 kJ/kg.K, and can be at least 2.30 kJ/kg.K. The present coolant composition also generally exhibits a thermal conductivity at 20° C. of at least 0.170 W/m.K, and even at least 0.200 W/m.K. Exhibiting such properties allows the coolant comprising the diester or triester species to be suitable for use in an electric vehicle. Overall, the present ester based coolant also has all the physical characteristics suitable for such use, including viscosity and pour point.

A process for operating an electric vehicle is therefore provided. The use of a proper coolant is vital to the operation of an electric vehicle. The use of the present ester based coolant in an electric vehicle allows for its operation.

The following examples are provided to demonstrate particular embodiments of the present invention. It should be appreciated by those of skill in the art that the methods disclosed in the examples which follow merely represent exemplary embodiments of the present invention. However, those of skill in the art should, in light of the present disclosure, appreciate that many changes can be made in the specific embodiments described and still obtain a like or similar result without departing from the spirit and scope of the present invention.

### EXAMPLES

Three diesters, A, B, and C, were prepared using the following olefins and carboxylic acids in accordance with the present process. The specific procedure for preparing diester A was as follows:

Tetradecenes were epoxidized as follows using a general procedure for the epoxidation of 7,8-tetradecene. To a stirred solution of 143 grams (0.64 mole) of 77% mCPBA (meta-chloroperoxybenzoic acid) in 500 mL chloroform, 100 grams (0.51 mol) of 7,8-tetradecene in 200 mL chloroform was added dropwise over a 45-minute period. The resulting reaction mixture was stirred overnight. The resulting milky solution was subsequently filtered to remove meta-chloro-benzoic acid that formed therein. The filtrate was then washed with a 10% aqueous solution of sodium bicarbonate. The organic layer was dried over anhydrous magnesium sulfate and concentrated on a rotary evaporator. The reaction afforded the desired epoxide (isomers of n-tetradecene epoxides) as colorless oil in 93% yield.

The isomers of n-tetradecene epoxides (10.6 grams, 50 mmol) were mixed with lauric acid (30 grams, 150 mmol) and 85% H3PO4 (0.1 grains, 0.87 mmol). The mixture was stirred and bubbled/purged with nitrogen at 150° C. for 20 hours. Excess lauric acid was removed from the product first by recrystallization in hexane with subsequent filtration at −15° C., and then by adding a calculated amount of 1N NaOH solution and filtering out the sodium laurate salt. The diester product collected (21.8 grams, 73% yield) was a light yellow, transparent oil. The oil comprised a mixture of diester species.

Diesters B and C were prepared using a similar procedure, but with the olefins and carboxylic acids noted below.

| Ester | Starting material-Olefin | Starting material-acid |
|---|---|---|
| A | C14 alpha olefin | Lauric acid |
| B | C14 alpha olefin | C6-C10 fatty acids |
| C | isomerized C16 olefin | C6-C10 fatty acids |

The three esters were evaluated for their electrical volume resistivity, pour point, specific heat and thermal conductivity characteristics. These were compared to other materials used as coolants. The results are shown in the Table below.

TABLE

| | Electrical | | | |
|---|---|---|---|---|
| | volume | | Specific | Thermal |
| | resistivity, | Pour | heat, | conductivity, |
| | 25° C. | point | 20° C. | 20° C. |
| | (Ohm-cm) | (° C.) | (kJ/kg · K) | (W/m · K) |
| Ester sample A | >10¹² | −27 | 2.07 | 0.178 |
| Ester sample B | >10¹⁰ | −60 | 2.03 | 0.208 |
| Ester sample C | >10¹⁰ | −53 | 2.39 | 0.196 |
| Perfluorocarbon | 10¹⁵ | −50 | 1.05 | 0.064 |
| Polydimethylsilicone | >10¹³ | <−50 | 1.46 | 0.150 |
| Alkyl benzene | >10¹² | −80 | 1.82 | 0.135 |
| Water/glycol 50/50 | 10⁶ | −45 | 3.31 | 0.416 |
| OAT Water/glycol | 10³ | −45 | 3.31 | 0.416 |
| 50/50 | | | | |

Table 1 summarizes the properties of the diester fluids in comparison with other base fluids used for coolant applications. The results in the table show that water/glycol mixtures are characterized by higher heat transfer properties in comparison with the non aqueous based fluids, but have much inferior electrical resistivities. The water/glycol mixture to which organic additive technology (OAT) has been added as a corrosion inhibitor package gives rise to an even lower electrical resistivity. For this latter reason the water/glycol based heat transfer fluids are not suitable as a heat transfer fluid where the dielectric properties like the electrical resistivity need to be high. The ester samples (A-C) have electrical resistivities significantly higher than the water/glycol mixtures and the magnitude of the electrical resistivity is of the order that it can be used as a dielectric fluid in applications with low conductive requirements

Various modifications and alterations of this invention will become apparent to those skilled in the art without departing from the scope and spirit of the invention. Other objects and advantages will become apparent to those skilled in the art from a review of the preceding description.

That which is claimed is:

1. A coolant for an electric vehicle comprising at least one diester or triester species having ester links on adjacent carbons.

2. The coolant of claim 1, wherein the coolant comprises a diester species.

3. The coolant of claim 2, wherein the diester species has the following structure: 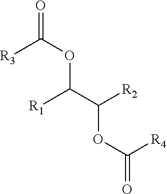
wherein R₁, R₂, R₃ and R₄ are the same or independently selected from hydrocarbon groups having from 2 to 17 carbon atoms.

4. The coolant of claim 2, wherein the diester species is derived from a process comprising:
a) epoxidizing an olefin having from about 8 to about 16 carbon atoms to form an epoxide comprising an epoxide ring;
b) opening the epoxide ring of step a) and forming a diol;
c) esterifying the diol of step b) with an esterifying species to form the diester species, wherein the esterifying species is selected from the group consisting of carboxylic acids, acyl halides, acyl anhydrides, and combinations thereof, wherein the esterifying species has a carbon number of from 2 to 18.

5. The coolant of claim 4, wherein the esterifying species is a carboxylic acid.

6. The coolant of claim 5, wherein the carboxylic acid is derived from a bio-derived fatty acid.

7. The coolant of claim 5, wherein the carboxylic acid is derived from alcohols generated by a Fischer-Tropsch process.

8. The coolant of claim 2, wherein the diester species is derived from a process comprising:
a) epoxidizing an olefin having from about 8 to about 16 carbon atoms to form an epoxide comprising an epoxide ring; and
b) reacting the epoxidized olefin with an esterifying species to form the diester species, wherein the esterifying species is selected from the group consisting of carboxylic acids, acyl halides, acyl anhydrides, and combinations thereof, wherein the esterifying species has a carbon number of from 2 to 18.

9. The coolant of claim 1, wherein the coolant comprises a triester species.

10. The coolant of claim 9, wherein the triester species has the following structure: 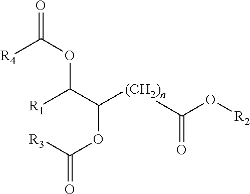
wherein R₁, R₂, R₃ and R₄ are the same or independently selected from hydrocarbon groups having from 2 to 20 carbon atoms and wherein “n” is an integer from 2 to 20.

11. The coolant of claim 9, wherein the triester species has the following structure: 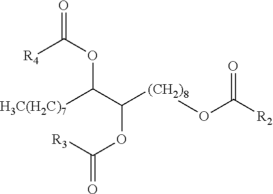
wherein R₂, R₃ and R₄ are typically the same or independently selected from C₂ to C₂₀ hydrocarbon groups.

12. The coolant of claim 9, wherein the triester species is derived from a process comprising:
a) esterifying a mono-unsaturated fatty acid having from 10 to 22 carbon atoms with an alcohol thereby forming an unsaturated ester;
b) epoxidizing the unsaturated ester in step a) thereby forming an epoxy-ester species comprising an epoxide ring;
c) opening the ring of the epoxy-ester species in step b) thereby forming a dihydroxy ester; and
d) esterifying the dihydroxy ester in step c) with an esterifying species to form a triester species, wherein the esterifying species is selected from the group consisting of carboxylic acids, acyl halides, acyl anhydrides, and combinations thereof, and wherein the esterifying species has a carbon number of from 2 to 19.

13. The coolant of claim 9, wherein the triester species is derived from a process comprising:
a) reducing a monosaturated fatty acid to the corresponding unsaturated alcohol;
b) epoxidizing the unsaturated alcohol to an epoxy fatty alcohol;
c) opening the ring of the epoxy fatty alcohol to make the corresponding triol; and
d) esterifying the triol of step c) with an esterifying species to form a triester species, wherein the esterifying species is selected from the group consisting of carboxylic acids, acyl halides, acyl anhydrides, and combinations thereof, and wherein the esterifying species has a carbon number of from 2 to 19.

14. The coolant of claim 9, wherein the triester species is derived from a process comprising:
a) reducing a monosaturated fatty acid to the corresponding unsaturated alcohol;
b) epoxidizing the unsaturated alcohol to an epoxy fatty alcohol;
c) esterifying the fatty alcohol epoxide with an esterifying species to form a triester species, wherein the esterifying species is selected from the group consisting of carboxylic acids, acyl halides, acyl anhydrides, and combinations thereof, and wherein the esterifying species has a carbon number of from 2 to 19.

15. The coolant of claim 1, wherein the coolant is for a hybrid electric vehicle.

16. The coolant of claim 1, wherein the coolant is for a battery in an electric vehicle.

17. The coolant of claim 1, wherein the coolant is for the cooling loop related to an electric motor and power electronics in an electric vehicle.

18. The coolant of claim 1, wherein the coolant exhibits an electrical volume resistivity at 25° C. of at least 10¹⁰ ohm-cm.

19. The coolant of claim 1, wherein the coolant exhibits an electrical volume resistivity at 25° C. of at least 10¹² ohm-cm.

20. The coolant of claim 1, wherein the coolant exhibits a specific heat at 20° C. of at least 2.00 kJ/kg.K.

21. The coolant of claim 1, wherein the coolant exhibits a specific heat at 20° C. of at least 2.30 kJ/kg.K.

22. The coolant of claim 1, wherein the coolant exhibits a thermal conductivity at 20° C. of at least 0.170 W/m.K.

23. The coolant of claim 1, wherein the coolant exhibits a thermal conductivity of at least 0.200 W/m.K.

24. The coolant of claim 1, wherein the coolant comprises a mixture of diester and triester species.

25. An electric vehicle comprising an electric motor with a cooling loop and a battery, with the coolant for the battery comprising the coolant of claim 1.

26. An electric vehicle comprising an electric motor with a cooling loop and a battery, with the coolant for the cooling loop comprising the coolant of claim 1.

27. An electric vehicle comprising an electric motor with a cooling loop and a battery, with the coolant for both the battery and the cooling loop comprising the coolant of claim 1.
